(19) United States
(12) Patent Application Publication
(10) Pub. No.: US 2010/0035853 A1
(43) Pub. Date: Feb. 11, 2010

(54) METHOD FOR PREVENTING OR TREATING CISPLATIN-INDUCED NEPHROTOXICITY

(75) Inventors: Haruyasu UEDA, Takarazuka-shi (JP); Haruki Okamura, Ibaraki-shi (JP)

Correspondence Address: SUGHRUE MION, PLLC, 2100 PENNSYLVANIA AVENUE, N.W., SUITE 800, WASHINGTON, DC, 20037, US

(73) Assignee: HYOGO COLLEGE OF MEDICINE, Nishinomiya-shi (JP)

(21) Appl. No.: 12/187,882

(22) Filed: Aug. 7, 2008

(51) Int. Cl. A61K 31/58; A61P 13/12

(52) U.S. Cl. 514/173

(57) ABSTRACT

Provided is a method for preventing or treating cisplatin induced nephrotoxicity, which comprises administering a patient who is receiving cisplatin a therapeutically effective amount of an aldosterone blocker such as eplerenone or spironolactone.

### TECHNICAL FIELD

The present invention relates to a method for preventing or treating Cisplatin induced nephrotoxicity. The present invention also relates to a method for treating cancer.

### BACKGROUND ART

Cisplatin, an effective chemotherapeutic agent used for treatment of various malignant tumors, is known to cause acute renal failure as a serious side effect (¹Leibbrandt, 1995; ²Schrier, 2002). Because of this side effect, the usage of Cisplatin was limited in the dose of administration or continuation of treatment. The mechanism for Cisplatin-induced renal toxicity is not sufficiently clarified and may be caused through several steps and pathways. The anti-cancer effect of Cisplatin may be caused by direct action of this drug on tubular cells forming cross-linkages of DNA. Cisplatin causes mitochondrial dysfunction (³Sugiyama, 1989; ⁴Nishikawa, 2001), generation of reactive oxygen species (ROS) (⁵ Matsushima, 1998), caspase activation (⁶ Kaushal, 2001), and inflammatory cell migration in the kidney (⁷Faubel, 2007; ⁸Lu, 2008). Moreover, Cisplatin induces production of cytokines such as TNF-α both in vivo (⁹Ramesh, 2002, ¹⁰Dong, 2007; ¹¹Zhang, 2007) and in vitro (¹²Tsuruya, 2003), and increased TNF-α may mediate Cisplatin-induced renal toxicity through induction of chemokines and cytokines (⁹Ramesh, 2002). Contrary to these, inhibition of TNF-α and deficiency of TNF-α were shown to attenuate renal toxicity caused by Cisplatin (⁹Ramesh, 2002; ¹⁰Dong, 2007). Many of the mediators in Cisplatin-induced renal toxicity are common with those involved in the renal failure caused by ischemia/reperfusion, and correspondingly, scavengers of ROS such as superoxide dismutase and Edaravone (¹³Davis, 2001; ¹⁴Sueishi, 2002), deficiency of caspase (¹⁵Faubel, 2004) were also shown to protect against Cisplatin-induced renal toxicity. IL-10, an anti-inflammatory cytokine, inhibits Cisplatin-induced renal toxicity by suppressing expression of TNF-α, ICAM-1 and inducible NO synthase (¹⁶Deng, 2001). Ligands for peroxisome proliferators-activated receptor ameliorate Cisplatin-induced renal toxicity, indicating the involvement of inflammatory reaction (¹⁷Li, 2004). In addition to above mediators, it has been recognized from early stage of Cisplatin therapy that Cisplatin causes abnormality in renin-angiotensin-aldosterone system (RAAS; ¹⁸Deegan, 1995), hyponatremia (¹⁹Hutchison, 1988) and the impairment of aldosterone receptor (²⁰Iida, 2000). Since chronic treatment of rats with aldosterone causes severe toxicity accompanying proteinuria (²¹Greene, 1996; ²²Blasi, 2003; ²³Nishiyama, 2004), and since inhibitors of RAAS attenuate renal toxicity in experimental hypertension model and diabetic model (²²Blasi, 2003; ²³Nishiyama, 2004; ²⁴Guo, 2006), it is probable that RAAS is involved in Cisplatin-induced renal toxicity.

Although IL-18 was originally found as an IFN-γ-inducing factor, it is a multifunctional cytokine up-regulating both Th1 and Th2 cytokines, activating NK cells, and augmenting Fas ligand (²⁵Nakanishi, 2001). IL-18 is produced by various cells and processed to active form through activation of caspase-1 (²⁶Ghayur, 1997). Oxidative stress or activation of inflammasome has been suggested to be concerned with its processing, but the mechanism of caspase-1 activation is not completely elucidated (²⁷Esposito, 2002; ²⁸Cruz, 2007; ²⁹Kummer, 2007; ³⁰Mariathasan, 2007). Recently, IL-18 was shown to play a crucial role in the mechanism for renal toxicity caused by ischemia/reperfusion through induction of IFN-γ (³¹Daemen, 1999) and augmentation of neutrophil infiltration to the kidney (³²Melnikov, 2001). However, it has also reported that IL-18 may play roles in renal toxicity by a mechanism independent of neutrophil (³³Melnikov, 2002). Since Cisplatin induces IL-18 in peripheral blood mononuclear cells, IL-18 also may play roles in Cisplatin-induced renal toxicity (⁷Faubel, 2007). In addition, IL-18 is abundantly included in the adrenal gland, which suggests the participation of IL-18 in immuno-adreno-cortical communication (³⁴Conti, 2000; ³⁵Bornstein, 2004).

### SUMMARY OF THE INVENTION

An object of the present invention to provide a novel method to prevent or treat the Cisplatin-induced nephrotoxicity. Another object of the present invention is to provide a method for preventing accumulation of Cisplatin in the kidney. A further object of the present invention is to provide a novel chemotherapy method using Cisplatin which causes reduced nephrotoxicity.

The instant inventors have found that IL-18 induced by Cisplatin stimulates the production of aldosterone, which in turn, prolongs the accumulation of Cisplatin in the kidney, and causes nephrotoxicity; and that the blockage of aldosterone receptor is effective for reducing nephrotoxicity induced by Cisplatin.

Accordingly, the instant application provides a method for preventing or treating a condition or disease caused by Cisplatin-induced nephrotoxicity, which comprises administering a therapeutically effective amount of an aldosterone blocker to a patient who is receiving Cisplatin.

According to the method of the present invention, Cisplatin and an aldosterone blocker may be administered to the patient simultaneously, sequentially, or separately, with or without additional agents or treatments, such as other anti-cancer agents other than Cisplatin or radiation therapy.

In another aspect, the present application provides a method for preventing accumulation of Cisplatin in the kidney of a patient, which comprises administering a therapeutically effective amount of an aldosterone blocker to said patient who is receiving Cisplatin.

In a further aspect, the instant application provides a method for treating caner, which comprises administering a therapeutically effective amount of Cisplatin and an aldosterone blocker to a patient in need thereof.

Preferred examples of aldosterone blockers may include Spironolactone and Eplerenone.

In still further aspect of the present invention, provided is a method for preventing or treating a condition or disease caused by Cisplatin-induced nephrotoxicity, which comprises administering a therapeutically effective amount of an IL-18 inhibitor to a patient who is receiving Cisplatin.

### BRIEF DESCRIPTION OF THE DRAWINGS

FIG. 1

### Elevation of Endogenous IL-18 in the Circulation by Cisplatin

Wild-type BALB/c mice were treated with Cisplatin (20 mg/kg, i.p.), then blood samples were collected at appropriate time (day) after Cisplatin treatment. Levels of serum IL-18 were measured using the specific ELISA kit. Each symbol and bar shows the mean value and the standard error obtained from 3 mice.

FIG. 2

Comparison of Survival Between Wild-Type and IL-18KO Mice After Treated with Cisplatin

Fourteen mice of both wild-type and IL-18KO mice were treated with Cisplatin (20 mg/kg, i.p.), then survived individuals were counted day by day. Open and closed symbols show wild-type and IL-18KO mice, respectively.

FIG. 3

### Cisplatin-Induced Increase in BUN and Creatinine in Wild-Type and IL-18KO Mice

Wild-type and IL-18KO mice were treated with Cisplatin (20 mg/kg, i.p.), then blood samples were collected at day 2 after Cisplatin treatment. Plasma levels of BUN and creatinine were measured as shown in Materials and Methods. Open and closed columns show vehicle (saline) and Cisplatin treatment, respectively. Each column and bar shows the mean value and the standard errors obtained from 3 mice. ** P<0.01; compared with vehicle (PBS concluding 0.5% normal mouse serum) treatment of respective mice (Student's T-test). NS; not significant.

FIG. 4

### Accumulation of Cisplatin in the Kidney

Wild-Type and IL-18KO Mice Were Treated with Cisplatin (20 mg/kg, i.p.), then pair of the kidney were isolated at appropriate time (day) after Cisplatin treatment. Accumulations of Cisplatin in the kidney were measured as platinum ion content in the kidney homogenate. Open and closed columns show wild-type and IL-18KO mice, respectively. Each symbol and bar shows the mean value and the standard error obtained from 3 mice. * P<0.05; compared with the value of wild-type mice at respective time point (Student's-T test).

FIG. 5

### Effect of Exogenously Supplemented IL-18 on Increase of BUN and Creatinine Induced by Cisplatin in IL-18KO Mice

Wild-type, IL-18KO and recombinant IL-18-supplemented IL-18KO mice were treated with Cisplatin (20 mg/kg, i.p.), then blood samples were collected at appropriate time after Cisplatin treatment. Plasma levels of BUN and creatinine were measured as shown in Materials and Methods. Open, closed and grey-colored columns show wild-type, IL-18KO and IL-18-supplemented IL-18KO mice, respectively. Each column and bar shows the mean value and the standard error obtained from 3 mice. * P<0.05 and *** P<0.001; compared with the value of wild-type mice at respective time point, # P<0.05 and ## P<0.01; compared between values of IL-18KO and IL-18-supplemented IL-18KO mice (Tukey's test for multiple comparison). NS; not significant.

FIG. 6

Plasma Aldosterone Level After Treatment with Cisplatin in Wild-Type and IL-18KO Mice

Wild-type and IL-18KO mice were treated with Cisplatin (20 mg/kg, i.p.), then blood samples were collected at appropriate time after Cisplatin treatment. Plasma levels of aldosterone were measured as shown in Materials and Methods. Open and closed columns show wild-type and IL-18KO mice, respectively. Each column and bar shows the mean value and the standard error obtained from 4 mice.

* P<0.05 and *** P<0.001; compared with the value at time 0 of respective type of mice. ## P<0.01; compared between values of wild and IL-18KO mice (Tukey's test for multiple comparison). NS; not significant.

FIG. 7

### Dose-Dependency of the Induction of Plasma Aldosterone Level Induced by Recombinant IL-18 in Wild-Type Mice

Wild-type mice were treated with various doses of IL-18 (0.2, 0.6 and 2 mg/mouse), then blood samples were collected at 5 hours after IL-18 treatment. Each column and bar shows the mean value and the standard error obtained from 3 mice. *** P<0.001; compared with the value at time 0 (Tukey's test for multiple comparison). NS; not significant.

FIG. 8

### Effect of Eplerenone on Cisplatin-Induced Increase in Plasma BUN and Creatinine in Wild-Type Mice

Wild-type mice were treated with vehicle (PBS) or Eplerenone for 30 min pre, and day 1 and day 2 post treatment of Cisplatin (20 mg/kg, i.p.), then blood samples were collected at appropriate time after Cisplatin treatment. Open and closed columns show vehicle- and Eplerenone-treated mice, respectively. Each column and bar shows the mean value and the standard error obtained from 3 mice. * P<0.05, *** P<0.001; compared values between vehicle (0.5% CMCaq) and Eplerenone treatment group at respective time point (Tukey's test for multiple comparison). NS; not significant.

FIG. 9

### Effect of Eplerenone on Cisplatin-Induced Increase in Plasma Aldosterone in Wild-Type Mice

Wild-type mice were treated with vehicle (PBS) or Eplerenone for 30 min pre, and day 1 and day 2 post treatment of Cisplatin (20 mg/kg, i.p.), then blood samples were collected at appropriate time after Cisplatin treatment. Open and closed columns show vehicle- and Eplerenone-treated mice, respectively. Each column and bar shows the mean value and the standard error obtained from 3-4 mice. ** P<0.01, *** P<0.001; compared values between vehicle (0.5% CMCaq) and Eplerenone treatment group at respective time point (Tukey's test for multiple comparison). NS; not significant.

FIG. 10

### Effect of Another Aldosterone Blocker Spironolactone on Cisplatin-Induced Increase in Plasma BUN and Creatinine in Wild-Type Mice

Wild-type mice were treated orally with vehicle (PBS), Spironolactone (10 mg/kg) or Eplerenone (20 mg/kg) for 30 min pre, and day 1 and day 2 post treatment of Cisplatin (20 mg/kg, i.p.), then blood samples were collected at 2 days after the Cisplatin treatment. Open, grey-colored and closed columns show vehicle- Spironolactone- and Eplerenone-treated mice, respectively. Each column and bar shows the mean value and the standard error obtained from 3 mice.

### BEST MODE FOR CARRYING OUT THE INVENTION

The present inventors has shown that IL-18 plays crucial roles in Cisplatin-induced acute renal failure. Cisplatin augmented production of IL-18 in the circulation as well as plasma BUN and creatinine, and IL-18KO mice were insensitive against the toxicity of Cisplatin, mortality and increase in plasma BUN and creatinine. In addition, exogenously supplemented IL-18 was also shown to reverse the decreased acute renal failure of Cisplatin in IL-18KO mice. These results suggest that IL-18 is involved in the exacerbation of Cisplatin-induced acute renal failure.

Cisplatin, once treated in a whole body of animals, has reported to be accumulated in the kidney and reveal its nephrotoxicity through production of reactive oxygen species (ROS) and activation of caspase-1 (⁴²Baek, 2003; ⁴³Stewart, 2007). It have already been reported that oxidative stress induces caspase-1 activation, and stimulates IL-18 production (⁴⁴Sekiyama, 2005). These evidences indicates that Cisplatin accumulate in the kidney, produce ROS and activate caspase-1, which in turn, stimulate IL-18 production. Furthermore, in the present study, the excretion of platinum ion was more rapid in IL-18KO mice than in wild-type mice, indicating that the accumulation of Cisplatin in the kidney might be mediated by IL-18. In the present application, IL-18 was shown to augment the plasma levels of aldosterone in a dose-dependent manner. In addition, plasma level of aldosterone induced by Cisplatin was raised in accordance with IL-18 production, and IL-18, when it is administered exogenously, stimulates aldosterone production in mice. Thus the inventors hypothesized the aldosterone induced by IL-18 might cause the accumulation of Cisplatin in the kidney via the anti-diuretic action, which in turn induce acute renal failure in mice treated with Cisplatin. This was paradoxically supported with the evidences that IL-18KO mice did not increase the levels of plasma BUN and creatinine after Cisplatin treatment and that these mice failed to produce aldosterone induced by Cisplatin. In the present study, selective receptor blockers for aldosterone, Eplerenone and Spironolactone strongly suppressed the renal toxicity of Cisplatin, indicating aldosterone plays a major role in the appearance of Cisplatin-induced acute renal failure. Furthermore, the inventors presented evidences that Cisplatin-induced aldosterone production was much lower in IL-18KO mice than in wild-type mice, and that renal excretion of platinum ion was more rapid in IL-18KO mice than in wild-type mice. Taken together these evidences, it is suggested that the renal accumulation of Cisplatin might be mediated by aldosterone induced by IL-18. The inventors has shown that the increase of plasma aldosterone induced by Cisplatin was reduced by the treatment with Eplerenone (FIG. 9).

In the specification and claims, the term “an aldosterone blocker” refers to an agent which can bind to receptors for mineralcorticoids to compete with the endogenous ligand aldosterone, and inhibit the it's signal transduction through the receptors. Examples of aldosterone blockers may include epilerenone and Spironolactone Among the above, Spironolactone and Eplerenone are especially useful in the present invention.

The expression “condition or disease caused by Cisplatin induced nephrotoxicity” includes renal failure, especially, acute renal failure.

The term “prevention”, “prevent” or “preventing” within the context of this invention refers not only to a complete prevention of a certain clinical condition, but also to any partial or substantial prevention, attenuation, reduction, decrease or diminishing of the condition before or at early onset of the condition or disease.

The term “treatment”, “treat” or “treating” within the context of this invention refers to any beneficial effect on progression of disease, including attenuation, reduction, decrease or diminishing of the pathological development after onset of condition.

The term “treat”, “treating” or “treatment” as used in relation to cancer, means reversing, alleviating, inhibiting the progress of, or preventing, either partially or completely, the growth of tumors, tumor metastases, or other cancer-causing or neoplastic cells in a patient.

The term “therapeutically effective amount” or “effective amount” means the amount of the compound or combination that will elicit the biological or medical response of a patient that is being sought by the veterinarian, medical doctor or other clinician.

The data presented in the Examples herein below demonstrated that an aldosterone blocker is effective for suppressing nephrotoxicity induced by Cisplatin administration. Accordingly, the present invention provides a method for reducing Cisplatin induced nephrotoxicity, comprising administering a therapeutically effective amount of an aldosterone blocker to a patient who is receiving Cisplatin.

According to the present invention, the aldosterone blocker may be administered to the patient simultaneously, sequentially or separately with Cisplatin.

The patient who is receiving Cisplatin may be a patient suffered from a disease or condition which can be treated by Cisplatin. The disease or condition may be any of those can be treated solely by Cisplatin or those can be treated by Cisplatin in combination with one or more anti-cancer agents other than Cisplatin or in combination of one or more anti-cancer treatment such as radiation and surgery. Examples of the conditions or diseases that can be treated by Cisplatin include various types of cancers and are disclosed in US2004/0258771, US2005/0271747, U.S. Pat. Nos. 6,251,355; 6,224,883; 6,130,245; 6,126,966; 6,077,545; 6,074,626; 6,046,044; 6,030,783; 6,001,817; 5,922,689; 4,322,391; and 4,310,515; the disclosures of which are herein incorporated by reference.

Examples of cancers to be treated by Cisplatin are lung cancer, colorectal cancer, NSCLC, bronchioloalviolar cell lung cancer, bone cancer, pancreatic cancer, skin cancer, cancer of the head or neck, cutaneous melanoma, intraocular melanoma, uterine cancer, ovarian cancer, rectal cancer, anal region cancer, stomach cancer, gastric cancer, colon cancer, breast cancer, uterine cancer, fallopian tube carcinoma, endometrial carcinoma, cervical carcinoma, vaginal carcinoma, vulval carcinoma, Hodgkin's Disease, esophagus cancer, small intestine cancer, endocrine system cancer, thyroid gland cancer, parathyroid gland cancer, adrenal gland cancer, soft tissue sarcoma, urethral cancer, penis cancer, prostate cancer, bladder cancer, kidney cancer, ureter cancer, renal cell carcinoma, renal pelvis carcinoma, mesothelioma, hepatocellular cancer, biliary cancer, chronic leukemia, acute leukemia, lymphocytic lymphoma, CNS neoplasm, spinal axis cancer, brain stem glioma, glioblastoma multiforme, astrocytoma, schwannoma, ependymoma, medulloblastoma, meningioma, squamous cell carcinoma and pituitary adenoma tumors or tumor metastases.

According to the present invention, the anti cancer agent other than Cisplatin may be any of known agents which have been used clinically in combination with Cisplatin for the treatment of the condition or disease to be treated by the method of the present invention.

The anti-cancer therapy which may be conducted simultaneously with administering cisplatin in the context of the present invention may be any of known treating procedures including radiotherapy and surgery.

Some examples of the anti-cancer agents as well as anti cancer treatment that can be carried out in combination with the cisplatin administration are disclosed in US2004/0258771, US2005/0271747, U.S. Pat. Nos. 6,251,355; 6,224,883; 6,130,245; 6,126,966; 6,077,545; 6,074,626; 6,046,044; 6,030,783; 6,001,817; 5,922,689; 4,322,391; and 4,310,515; the disclosures of which are herein incorporated by reference.

According to the present invention, Cisplatin is typically administered to the patient in a dose regimen that provides for the most effective treatment of the disease or condition for which the patient is being treated, as known in the art. Since the nephrotoxicity of Cisplatin and deposition of Cisplatin in the kidney will be suppressed by the method of the present invention, the amount of Cisplatin to be administered to the patient may be higher than the amount currently instructed by the medical doctor or other clinical practitioner. Any of dosage forms of Cisplatin available on the market may be used for the method of the present invention.

Before, during and/or after cisplatin administration, an extra fluid, such as saline, infusion and/or diuretic may be given to the patient.

According to the present invention, the patient may be those who have already been receiving Cisplatin prior to the start of the method of the present invention or those who is going to receive Cisplatin.

In the method of the present invention, the amount and the timing of the aldosterone blocker administration may vary depending on the type, such as age, weight and gender and condition of the patient being treated, the severity of the disease or condition being treated, and on the route of administration. In addition, the administration of the aldosterone blocker will also be determined depending on the amount and timing of the co-administering Cisplatin and the other anti-cancer agent and/or anti cancer treatment.

Administration of the aldosterone blocker may be accomplished by oral route, or by intravenous, intramuscular or subcutaneous injections. The formulation may be in the form of a bolus, or in the form of aqueous or non-aqueous isotonic sterile injection solutions or suspensions. These solutions and suspensions may be prepared from sterile powders or granules having one or more pharmaceutically-acceptable carriers or diluents, or a binder such as gelatin or hydroxypropyl-methyl cellulose, together with one or more of a lubricant, preservative, surface-active or dispersing agent.

Typically, the aldosterone blocker is administered in a daily dose ranging from about 1 to about 2000 mg, especially, once daily about 50-200 mg of eprelenone is administered orally for an adult male patient.

For oral administration, the pharmaceutical composition may be in the form of, for example, a tablet, capsule, suspension or liquid. The pharmaceutical composition is preferably made in the form of a dosage unit containing a particular amount of the active ingredient. The active ingredients may also be administered by injection as a composition wherein, for example, saline, dextrose or water may be used as a suitable carrier.

According to a typical example of the present invention, when the patient to be treated is a person who is suffered from testicular cancer, cervical cancer, prostate cancer, bladder cancer, esophageal cancer, ovarian cancer, stomach cancer, neuroblastoma, or non-small cell lung cancer and is receiving once daily infusion of 15-20 mg/m² body surface area of Cisplatin for 5 successive days followed by two weeks withdrawal per treatment cycle, the patient may be administered orally with 50-200 mg of Eprelenone once daily during the period of receiving Cisplatin. Eprelenone may be given to the patient before, simultaneously, or after each infusion of Cisplatin. The patient may also be administered with a diuretic and/or an extra fluid infusion.

The present invention also provides a method for preventing and treating a condition caused by Cisplatin induced nephrotoxicity, which comprises administering an IL-18 inhibitor such as anti-IL-18 antibody to a patient who is receiving Cisplatin.

The further details of the present invention will follow with reference to test examples, which, however, are not intended to limit the present invention.

### EXAMPLES

### Materials and Methods

### Animals

Wild-type male BALB/c mice (WT mice) aged 9-10 weeks-old were purchased from SHIMIZU Laboratory Supplies Co., Ltd. (Kyoto, Japan). Age-matched BALB/c-background mice deficient for IL-18 gene (IL-18KO mice) were raised by backcrossing for >8 generations in National Institute for Agrobiological Sciences (Tsukuba, Ibaraki, Japan). Homozygous mutant mice were used for breeding and experiments in our animal facilities. These mice were kept in air-conditioned rooms at 24±2° C. and given tapped water and solid food (MF, Charles-River Japan) ad libitum. All experimental procedures in this experiment were approved by the Animal Care Committee of Hyogo College of Medicine.

### Reagents

Cisplatin was purchased from Sigma (St. Louis, Mo., USA). Recombinant mouse IL-18 (rmIL-18) and Eplerenone were kindly donated by GlaxoSmithKline Pharmaceuticals (PA, USA) and Pfizer Inc (NY, USA), respectively. Spironolactone was purchased from Sigma (St. Louis, Mo., USA).

### Induction of Renal Toxicity by Cisplatin

Cisplatin, dissolved in warmed saline at a concentration of 1 mg/ml. In order to induce renal toxicity, Cisplatin were given with a single intraperitoneal injection of a toxic dose (20 mg/kg). As a control, mice were treated with a similar volume of saline. Some mice were also received recombinant mouse IL-18 (2 μg/body) prior to Cisplatin. Eplerenone, dissolved with 0.5% CMCaq, was treated orally with a dose of 20 mg/kg for 3 times at 30 min prior to Cisplatin, day 1 and day 2. Spironolactone dissolved with 0.5% CMCaq, was treated orally with a dose of 10 mg/kg for 3 times at 30 min prior to Cisplatin, day 1 and day 2.

### Measurement of Blood Urea Nitrogen, Creatinine and Aldosterone

Blood taken from mice treated with or without Cisplatin were centrifuged, and plasma samples were collected and frozen at −80° C. until use. Blood urea nitrogen (BUN) and creatinine were enzymatically measured by HITACHI 7180 auto-analyzer. Aldosterone level was measured by radioimmuno assay. All of these assays were performed by SRL, Inc (Tokyo, Japan).

### Measurement of Serum Levels of Cytokines

IL-18 concentrations in serum were measured by ELISA purchased from Medical and Biological Laboratories (Tokyo, Japan) in accordance with the manufactures' instructions. Other cytokines such as TNF-α, IFN-γ, IL-1β, IL-10 and MIP-1α were measured respective ELISA kits purchased from R&D Systems Inc (USA).

### Statistical Analysis

Data were analyzed by Student's-T test or Tukey's test for multiple comparisons. A P value less than 0.05 was considered to be a statistically significant difference. Results were expressed as the mean±SE of the repeated experiments.

### Results

### Elevation of Endogenous IL-18 in the Circulation by Cisplatin

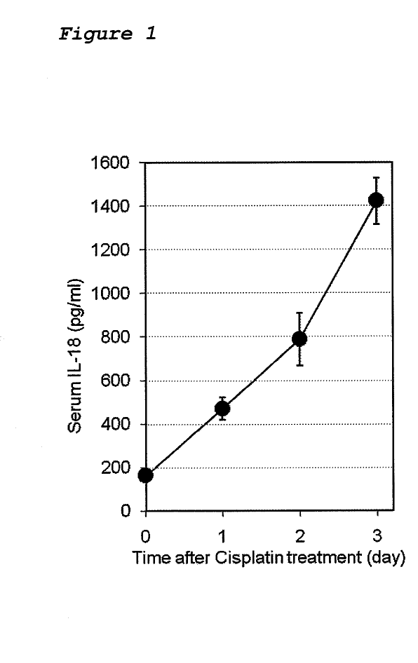

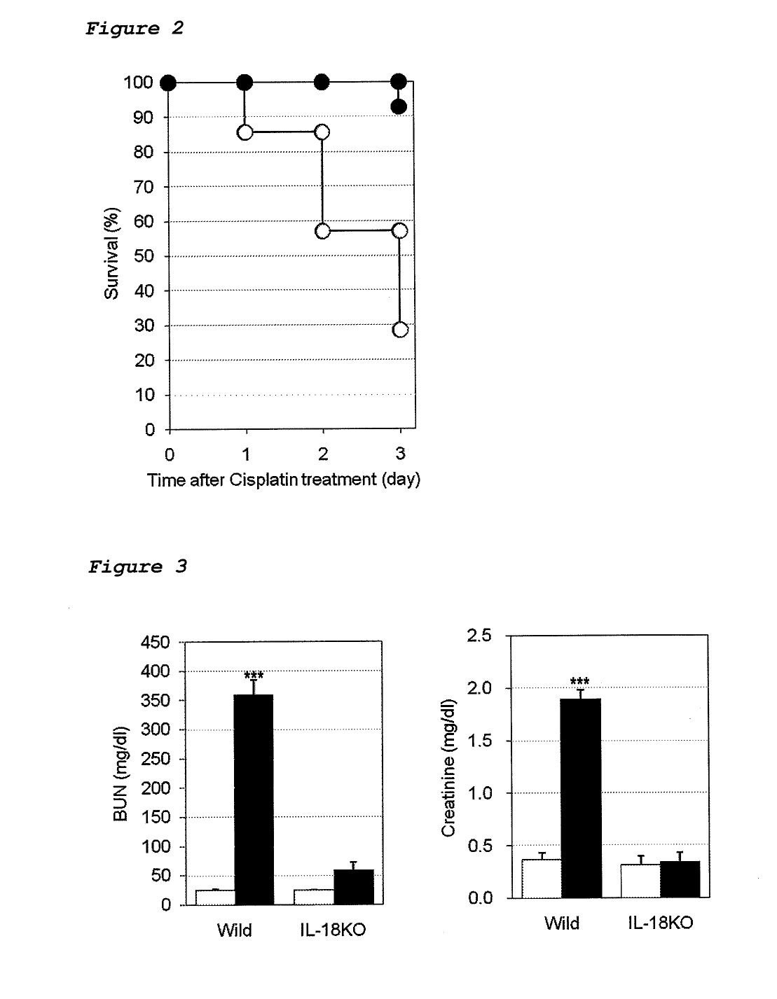

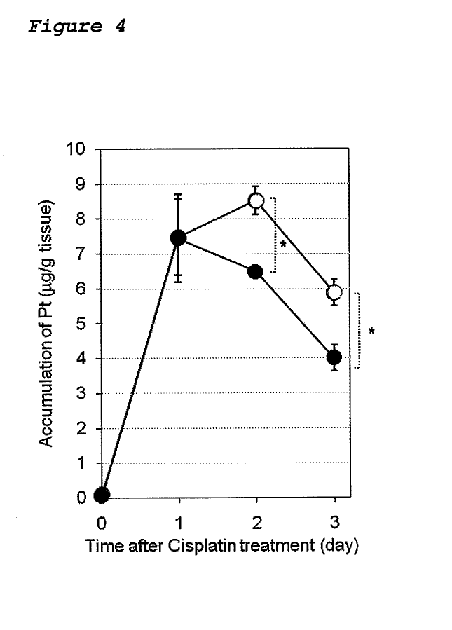

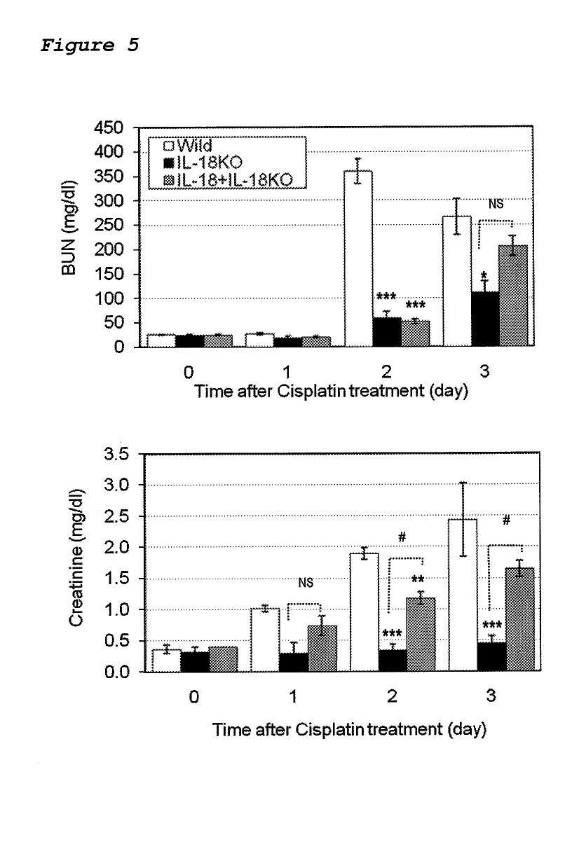

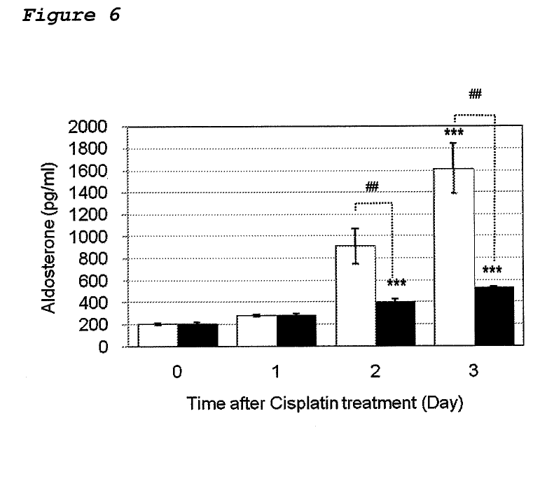

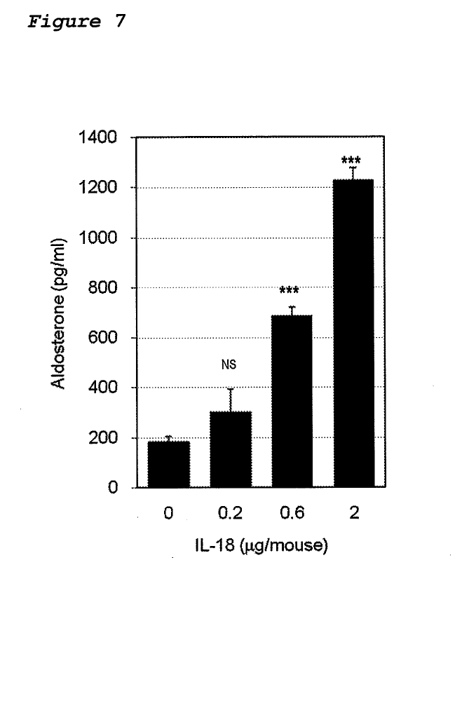

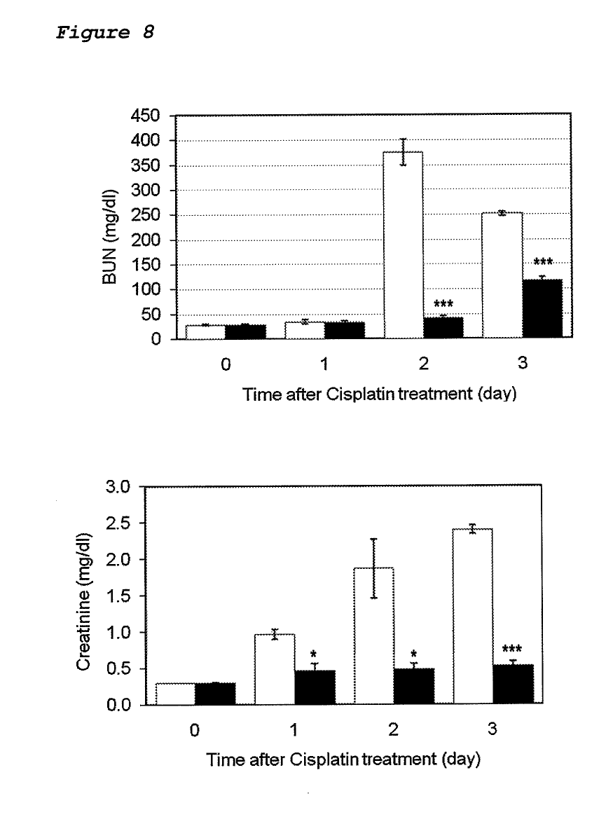

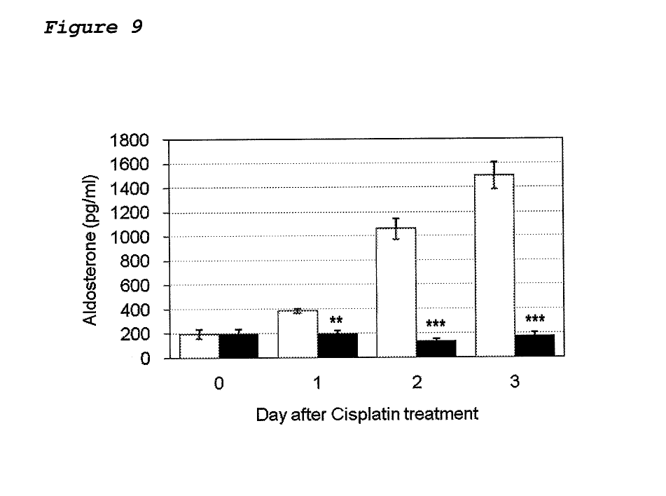

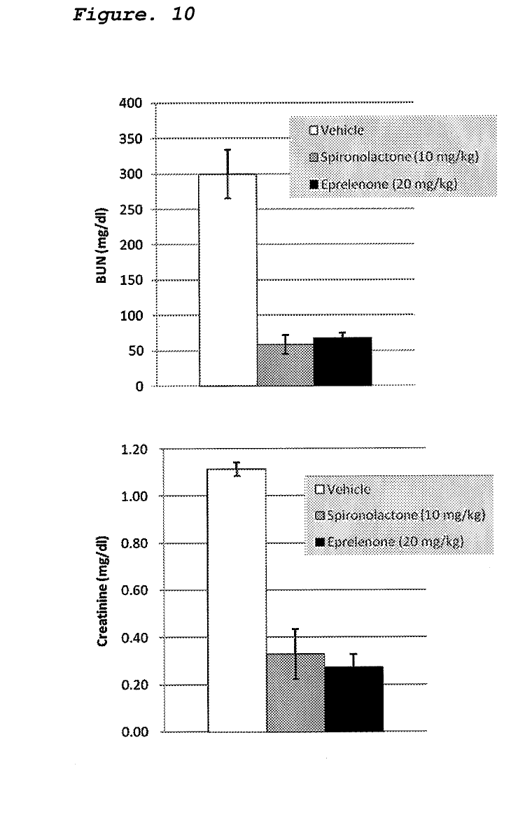

When Cisplatin at a concentration of toxic dose (20 mg/kg, i.p.) was administered to wild-type of BALB/c mice, we found that levels of IL-18 in the circulation were markedly raised day by day, and reached to the level over 1000 pg/ml at around day 3 after the administration of Cisplatin (FIG. 1). Other cytokines such as TNF-α, IFN-γ, IL-1β, IL-10 and MIP-1α0 were undetectable when IL-18 was detected at the maximal levels in the present study (data not shown).

### Decreased Mortality in IL-18KO Mice

In order to examine the involvement of IL-18 in the toxicity of Cisplatin, the effect of Cisplatin on the survival of mice was compared between wild-type and IL-18KO of BALB/c background mice. By the administration of higher dose, toxicity of Cisplatin was induced. First dead mice were observed in wild-type mice at day 1 after administration of Cisplatin. Then, six of fourteen mice were dead in wild-type mice, while no mouse was dead in IL-18KO mice, at day 2 after administration of Cisplatin. The survival rate was 28.6% (4 of 14) in wild-type mice and 92.9% (13 of 14) in IL-18 KO mice at day 3 after administration of Cisplatin (FIG. 2).

### Attenuated Acute Renal Failure Induced by Cisplatin in IL-18 KO Mice

Cisplatin shows strong acute renal failure only in wild-type mice in this examination, and the toxicity of Cisplatin was clearly observed at day 2 after administration. Therefore, we measured plasma markers of renal function, blood urea nitrogen (BUN) and creatinine, at day 2 after Cisplatin administration, and compared between wild-type and IL-18KO mice. As shown in FIG. 3 left panel, plasma levels of BUN were significantly induced by the administration of Cisplatin in wild-types of mice, however, it was significantly lowered in IL-18KO mice than in wild-type mice. In addition, plasma levels of creatinine were also significantly lower in IL-18KO mice than in wild-type mice with the almost same way as shown in BUN (FIG. 3, right panel).

### Measurement of the Accumulation of Cisplatin in the Kidney

Incidence of acute renal failure by Cisplatin has known to be caused by the accumulation of Cisplatin in the kidney. Then, we measured the amount of the accumulation of platinum ion in the kidney, and compared it between wild-type and IL-18KO mice. The quantity of platinum ion in the kidney of both types of mice was almost equivalent at day 1 after Cisplatin treatment, however, the rate of excretion of platinum ion was clearly much rapid in IL-18KO mice than in wild-type mice at day 2 and 3 after Cisplatin treatment (FIG. 4).

Restoration of Sensitivity to Cisplatin in IL-18KO Mice by the Exogenous Treatment with Recombinant Mouse IL-18

To confirm the pathogenic role of IL-18 in Cisplatin-induced acute renal failure, recombinant mouse (rm) IL-18 was given to IL-18KO mice in the simultaneous treatment with Cisplatin. As shown in FIG. 5 upper panel, plasma levels of BUN in IL-18KO mice supplemented with rmIL-18 were increased to almost equivalent levels with wild-type mice at day 3 after Cisplatin treatment. Furthermore, plasma levels of creatinine induced by Cisplatin were also significantly increased by the supplement of rmIL-18 to IL-18KO mice at day 1 to 3 after Cisplatin treatment (FIG. 5, lower panel).

Plasma Aldosterone Level After Treatment with Cisplatin in Wild-Type and IL-18KO Mice

We observed that plasma aldosterone was increased day by day after administration of Cisplatin in wild-type mice. On the contrary, the induction of plasma levels of aldosterone was apparently lower in IL-18KO than in wild-type mice, but significant increases were observed at day 2 and 3 after Cisplatin treatment in IL-18KO mice (FIG. 6). Significant differences in the Cisplatin-induced increase in plasma aldosterone levels between wild-type and IL-18KO mice were observed at day 2 and 3 after Cisplatin treatment (FIG. 6).

### Augmentation of Plasma Levels of Aldosterone by IL-18

Since it was suggested that IL-18 is mediating the raise of plasma aldosterone and since aldosterone was suggested to have pathogenic roles in renal toxicity in several cases, effect of exogenously given IL-18 on plasma levels of aldosterone was examined. IL-18, given at various doses, augmented the plasma levels of aldosterone in a dose-dependent manner, and significant induction was observed over 0.6 μg of IL-18 (FIG. 7). However, notably, exogenously given IL-18 alone did not cause any renal toxicity in these mice (data not shown).

### Prevention of Cisplatin-Induced Acute Renal Failure by the Blockade of Aldosterone Receptor

Since production of aldosterone was raised by Cisplatin and IL-18, the effect of blockade of aldosterone receptor was examined on Cisplatin-induced acute renal failure (FIG. 8). Administration of Eplerenone, a recently developed selective blocker for aldosterone receptors, almost completely suppressed the elevation of plasma BUN and creatinine (FIG. 8). Significant inhibition of increased BUN level by Eplerenone was observed at day 2 and 3 after Cisplatin treatment (FIG. 8, upper panel). In case of creatinine, Eplerenone treatment was more sensitive and significant inhibition of increased creatinine level was observed at day 1 and later (FIG. 8, lower panel). In parallel with this, the lethality of Cisplatin also almost completely suppressed in mice treated with Eplerenone (data not shown).

### Prevention of Cisplatin-Induced Increase in Plasma Aldosterone Level by Eplerenone

As shown in FIG. 8, Eplerenone prevented acute renal failure induced by Cisplatin. In addition to this, we found that plasma level of aldosterone was also reduced by Eplerenone. Surprisingly, Eplerenone completely inhibited the increase in plasma aldosterone level induced by Cisplatin during observation for 3 days, and significant inhibition of plasma aldosterone increase by Eplerenone was observed at day 1 and later after Cisplatin treatment (FIG. 9).

### Effect of Another Aldosterone Blocker Spironolactone on Cisplatin-Induced Increase in Plasma BUN and Creatinine Levels

In order to confirm whether the prevention of Cisplatin-induced renal toxicity was dependent upon the blockade of aldosterone receptor, we examined using another aldosterone blocker Spironolactone. As shown in FIG. 10, Spironolactone also showed equipotent effect to Eplerenone in reducing plasma levels of BUN and creatinine increased after Cisplatin treatment at day 2. This result suggests that the blockade of an aldosterone receptor specifically reduce the renal toxicity induced by Cisplatin.

### REFERENCES

The following references are herein incorporated by reference.
1. Leibbrandt, M. E., Wolfgang, G. H., Metz, A. L., Ozobia, A. A., and Haskins, J. R. 1995. Critical subcellular targets of cisplatin and related platinum analogs in rat renal proximal tubule cells. Kidney Int. 48:761-770.
2. Schrier, R. W. 2002. Cancer therapy and renal injury. J. Clin. Invest. 110:743-745.
3. Sugiyama, S., Hayakawa, M., Kato, T., Hanaki, Y., Shimizu, K., and Ozawa, T. 1989. Adverse effects of anti-tumor drug, cisplatin, on rat mitochondria: disturbances in glutathione peroxidase activity. Biochem. Biophys. Res. Commun. 159:1121-1127.
4. Nishikawa, M., Nagatomi, H., Chang, B. J., Sato, E., and Inoue, M. 2001. Targeting superoxide dismutase to renal proximal tubule cells inhibits mitochondrial injury and renal dysfunction induced by cisplatin. Arch. Biochem. Biophys. 387:78-84.
5. Matsushima, H., Yonemura, K., Ohishi, K., and Hishida, A. 1998. The role of oxygen free radicals in cisplatin-induced acute renal failure in rats. J. Lab. Clin. Med. 131:518-526.
6. Kaushal, G. P., Kaushal, V., Hong, X., and Shah, S. V. 2001. Role and regulation of activation of caspases in cisplatin-induced injury to renal tubular epithelial cells. Kidney int. 60:1726-1736.
7. Faubel, S., Lewis, E. C., Reznikov, L., Ljubanovic, D., Hoke, T. S., Somerset, H., Oh, D. J., Lu, L., Klein, C. L., Dinarello, C. A., et al. 2007. Cisplatin-induced acute renal failure is associated with an increase in the cytokines interleukin (IL)-1beta, IL-18, IL-6, and neutrophil infiltration in the kidney. J. Pharmacol. Exp. Ther. 322:8-15.
8. Lu, L. H., Oh, D. J., Durson, B., He, Z., Hoke, T. S., Faubel, S., and Edelstein, C. L. 2008. Increased macrophage infiltration and fractalkine expression in Cisplatin-induced acute renal failure in mice. J. Pharmacol. Exp. Ther. 324:111-117.
9. Ramesh, G. and Reeves, W. B. 2002. TNF-alpha mediates chemokine and cytokine expression and renal injury in cisplatin nephrotoxicity. J. Clin. Invest. 110:835-842.
10. Dong, Z., and Atherton, S. S. 2007. Tumor necrosis factor-alpha in Cisplatin nephrotoxicity: a homebred foe? Kidney Int. 72:5-7.
11. Zhang, B., Ramesh, G., Norbury, C. C., and Reeves, W. B. 2007. Cisplatin-induced nephrotoxicity is mediated by tumor necrosis factor-alpha produced by renal parenchymal cells. Kidney Int. 72: 37-44.
12. Tsuruya, K., Ninomiya, T., Tokumoto, M., Hirakawa, M., Matsutani, K., Taniguchi, M., Fukuda, K., Kanai, H., Kishihara, K., Hirakata, H., et al. 2003. Direct involvement of the receptor-mediated apoptotic pathways in cisplatin-induced renal tubular cell death. Kidney Int. 63:72-82.
13. Davis, C. A., Nick, H. S., and Agarwal, A. 2001. Manganese superoxide dismutase attenuates Cisplatin-induced renal injury: importance of superoxide. J. Am. Soc. Nephrol. 12:2683-2690.
14. Sueishi, K., Mashima, K., Makino, K., Itoh, Y., Tsuruya, K., Hirakata, H., and Oishi, R. 2002. Protection by a radical scavenger edaravone against cisplatin-induced nephrotoxicity in rats. Eur. J. Pharmacol. 451:203-208.
15. Faubel, S., Ljubanovic, D., Reznikov, L., Somerset, H., Dinarello, C. A., and Edelstein, C. L. 2004. Caspase-1-deficient mice are protected against cisplatin-induced apoptosis and acute tubular necrosis. Kidney Int. 66:2202-13.
16. Deng, J., Kohda, Y., Chiao, H., Wang, Y., Hu, X., Hewitt, S. M., Miyaji, T., McLeroy, P., Nibhanupudy, B., Li, S., et al. 2001. Interleukin-10 inhibits ischemic and cisplatin-induced acute renal injury. Kidney Int. 60:2118-2128.
17. Li, S., Basnakian, A., Bhatt, R., Megyesi, J., Gokden, N., Shah, S. V., and Portilla, D. 2004. PPAR-alpha ligand ameliorates acute renal failure by reducing cisplatin-induced increased expression of renal endonucrease G. Am. J. Physiol. Renal Physiol. 287:F990-998.
18. Deegan, P. M., Nolan, C., Ryan, M. P., Basinger, M. A., Jones, M. M., and Hande, K. R. 1995. The role of the renin-angiotensin system in cisplatin nephrotoxicity. Renal Fail. 17:665-674.
19. Hutchison, F. N., Perez, E. A., Gandara, D. R., Lawrence, H. J., and Kaysen, G. A. 1988. Renal salt wasting in patients treated with cisplatin. Ann. Intern. Med. 108:21-25.
20. Iida, T., Makino, Y., Okamoto, K., Yoshikawa, N., Makino, I., Nakamura, T., and Tanaka, H. 2000. Functional modulation of the mineralocorticoid receptor by cis-diamminedichloroplatinum (II). Kidney Int. 58:1450-1460.
21. Greene, E. L., Kren, S., and Hostetter, T. H. 1996. Role of aldosterone in the remnant kidney model in the rat. J. Clin. Invest. 98:1063-1068.
22. Blasi, E. R., Rocha, R., Rudolph, A. E., Blomme, E. A., Polly, M. L., and McMahon, E. G. 2003. Aldosterone/salt induces renal inflammation and fibrosis in hypertensive rats. Kidney Int. 63:1791-1800.
23. Nishiyama, A., Yao, L., Nagai, Y., Miyata, K., Yoshizumi, M., Kagami, S., Kondo, S., Kiyomoto, H., Shokoji, T., Kimura, S., et al. 2004. Possible contributions of reactive oxygen species and mitogen-activated protein kinase to renal injury in aldosterone/salt-induced hypertensive rats. Hypertension. 43:841-848.
24. Guo, C., Martinez-Vasquez, D., Mendez, G. P., Toniolo, M. F., Yao, T. M., Oestreicher, E. M., Kikuchi, T., Lapointe, N., Pojoga, L., Williams, G. H., et al. 2006. Mineralocorticoid receptor antagonist reduces renal injury in rodent models of types 1 and 2 diabetes mellitus. Endocrinology. 147:5363-5373.
25. Nakanishi, K., Yoshimoto, T., Tsutsui, H., and Okamura, H. 2001. Interleukin-18 regulates both Th1 and Th2 responses. Annu. Rev. Immunol. 19:423-474.
26. Ghayur, T., Banerjee, S., Hugunin, M., Butler, D., Herzog, L., Carter, A., Quintal, L., Sekut, L., Talanian, R., Paskind, M., et al. 1997. Caspase-1 processes IFN-gamma-inducing factor and regulates LPS-induced IFN-gamma production. Nature. 386:619-623.
27. Esposito, K., Nappo, F., Marfella, R., Giugliano, G., Giugliano, F., Ciotola, M., Quagliaro, L., Ceriello, A., and Giugliano, D. 2002. Inflammatory cytokine concentrations are acutely increased by hyperglycemia in humans: role of oxidative stress. Circulation. 106:2067-2072.
28. Cruz, C. M., Rinna, A., Forman, H. J., Ventura, A. L., Persechini, P. M., and Ojcius, D. M. 2007. ATP activates a reactive oxygen species-dependent oxidative stress response and secretion of proinflammatory cytokines in macrophages. J. Biol. Chem. 282:2871-2879.
29. Kummer, J. A., Broekhuizen, R., Everett, H., Agostini, L., Kuijk, L., Martinon, F., van Bruggen, R., and Tschopp, J. 2007. Inflammation components NALP 1 and 3 show distinct but separate expression profiles in human tissues suggesting a site-specific role in the inflammatory response. J. Histochem. Cytochem. 55:443-452.
30. Mariathasan, S., and Monack, D. M. 2007. Inflammasome adaptors and sensors: intracellular regulators of infection and inflammation. Nat. Rev. Immunol. 7:31-40.
31. Daemen, M. A., van't Veer, C., Wolfs, T. G., and Buurman, W. A. 1999. Ischemia/reperfusion-induced IFN-gamma up-regulation: Involvement of IL-12 and IL-18. J. Immunol. 162:5506-5510.
32. Melnikov, V. Y., Ecder, T., Fantuzzi, G., Siegmund, B., Lucia, M. S., Dinarello, C. A., Schrier, R. W., and Edelstein, C. L. 2001. Impaired IL-18 processing protects caspase-1-deficient mice from ischemic acute renal failure. J. Clin. Invest. 107:1145-1152.
33. Melnikov, V. Y., Faubel, S., Siegmund, B., Lucia, M. S., Ljubanovic, D., and Edelstein, C. L. 2002. Neutrophil-independent mechanisms of caspase-1 and IL-18-mediated ischemic acute tubular necrosis in mice. J. Clin. Invest. 110:1083-1091.
34. Conti, B., Sugama, S., Kim, Y., Tinti, C., Kim, H., Baker, H., Volpe, B., Attardi, B., and Joh, T. 2000. Modulation of IL-18 production in the adrenal cortex following acute ACTH or chronic corticosterone treatment. Neuroimmunomodulation. 8:1-7.
35. Bornstein, S. R., Rutkowski, H., and Vrezas, I. 2004. Cytokines and steroidogenesis. Mol. Cell. Endocrinol. 215:135-141.
36. Kostova, I. 2006. Platinum complexes as anticancer agents. Recent Patents Anticancer Drug Discov. 1:1-22.
37. Arany, I., and Safirstein, R. L. 2003. Cisplatin nephrotoxicity. Semin. Nephrol. 23:460-464.
38. Ramesh, G., Zhang, B., Uematsu, S., Akira, S., and Reeves, W. B. 2007. Endotoxin and cisplatin synergistically induce renal dysfunction and cytokine production in mice. Am. J. Physiol. Renal Physiol. 293:F325-F332.
39. Okamura, H., Tsutsui, H., Kashiwamura, S, Yoshimoto, T., and Nakanishi, K. 1998. Interleukin-18: a novel cytokine that augments both innate and acquired immunity. Adv. Immunol. 70:281-312.
40. Ogura, T., Ueda, H., Hosohara, K., Tsuji, R., Nagata, Y., Kashiwamura, S., and Okamura, H. 2001. Interleukin-18 stimulates hematopoietic cytokines and growth factor formation and augments circulating granulocytes in mice. Blood. 98:2101-2107.
41. Ueda, H., Kashiwamura, S., Sekiyama, A., Ogura, T., Gamachi, N., and Okamura, H. 2006. Production of IL-13 in spleen cells by IL-18 and IL-12 through generation of NK-like cells. Cytokine. 33:179-187.
42. Baek, S. M., Kwon, C. H., Kim, J. H., Woo, J. S., Jung, J. S., and Kim, Y. K. 2003. Differential roles of hydrogen peroxide and hydroxyl radical in Cisplatin-induced cell death in renal proximal tubular epithelial cells. J. Lab Clin. Med. 142:178-186.
43. Stewart, J. H. 4th., Tran, T. L., Levi, N., Tsai, W. S., Schrump, D. S., and Nguyen, D. M. 2007. The essential role of the mitochondria and reactive oxygen species in Cisplatin-mediated enhancement of fas ligand-induced apoptosis in malignant pleural mesothelioma. J. Surg. Res. 141:120-131.
44. Sekiyama, A., Ueda, H., Kashiwamura, S., Sekiyama, R., Takeda, M., Rokutan, K., and Okamura, H. 2005. A stress-induced, superoxide-mediated caspase-1 activation pathway causes plasma IL-18 upregulation. Immunity. 22:669-677.
45. Kurt, E., Manavogle, O., Dilek, K., Orhan, B., and Evrensel, T. 1999. Effect of Cisplatin on plasma rennin activity and serum aldosterone levels. Clin. Nephrol. 52:397-398.
46. Mejia-Vilet, J. M., Ramirez, V., Cruz, C., Uribe, N., Gamba, G., and Bobadilla, N. A. 2007. Renal ischemia-reperfusion injury is prevented by the mineralocorticoid receptor blocker spironolactone. Am. J. Physiol. Renal Physiol. 293:F78-F86.
47. Seta, Y., Kanda, T., Tanaka, T., Arai, M., Sekiguchi, K., Yokoyama, T., Kurimoto, M., Tamura, J., and Kurabayashi, M. 2000. Interleukin-18 in patients with congestive heart failure: induction of atrial natriuretic peptide gene expression. Res. Commun. Mol. Pathol. Pharmacol. 108:87-95.
48. Tan, A. C., Russel, F. G., Thien, T., and Benraad, T. J. 1993. Atrial natriuretic peptide. An overview of clinical pharmacology and pharmacokinetics. Clin. Pharmacokinet. 24:28-45.
49. Corti, R., Burnett, J. C., Rouleau, J. L., Ruschitzka, F., and Lüscher, T. F. 2001. Vasopeptidase inhibitors: a new therapeutic concept in cardiovascular disease? Circulation. 104:1856-1862.
50. Sarzani, R., Opocher, G., Paci, M. V., Belloni, A. S., Mantero, F., Dessi-Fulgheri, P., and Rappelli, A. 1999. Natriuretic peptides receptors in human aldosterone-secreting adenomas. J. Endocrinol. Invest. 22:514-518.
51. Cherradi, N., Brandenburger, Y., Rossier, M. F., Vallotton, M. B., Stocco, D. M., and Capponi, A. M. 1998. Atrial natriuretic peptide inhibits calcium-induced steroidogenic acute regulatory protein gene transcription in adrenal glomerulosa cells. Mol. Endocrinol. 12: 962-972.
52. Ito, T., Yoshimura, M., Nakamura, S., Nakayama, M., Shimasaki, Y., Harada, E., Mizuno, Y., Yamamuro, M., Harada, M., Saito, Y., et al. 2003. Inhibitory effect of natriuretic peptides on aldosterone synthase gene expression in cultured neonatal rat cardiocytes. Circulation. 107:807-810.

What is claimed is:

1. A method for preventing or treating a condition or disease caused by Cisplatin-induced nephrotoxicity, which comprises administering a therapeutically effective amount of an aldosterone blocker to a patient who is receiving Cisplatin.

2. The method of claim 1, wherein the patient is a human being treated for cancer.

3. The method of claim 1, wherein the aldosterone blocker and Cisplatin are co-administered to the patient in different formulations.

4. The method of claim 1, wherein aldosterone blocker is Spironolactone or Eplerenone.

5. The method of claim 1, wherein the patient is receiving one or more anti-cancer agents other than Cisplatin.

6. The method of claim 1, wherein the patient is receiving radiation.

7. The method of claim 1, wherein the condition caused by Cisplatin-induced nephrotoxicity is acute renal failure.

8. A method for preventing accumulation of Cisplatin in the kidney, which comprises administering a therapeutically effective amount of an aldosterone blocker to a patient who is receiving Cisplatin.

9. A method for treating caner, which comprises administering a therapeutically effective amount of Cisplatin and an aldosterone blocker to a patient in need thereof.
